(19) United States
(12) Patent Application Publication
(10) Pub. No.: US 2016/0073925 A1
(43) Pub. Date: Mar. 17, 2016

(54) Obtaining a Three-dimensional Image of a Medical Instrument with a Magnetic Resonance Tomography Device

(71) Applicants: David Grodzki, Erlangen (DE); Annemarie Hausotte, Erlangen (DE); Björn Heismann, Erlangen (DE); Arne Hengerer, Möhrendorf (DE); Mark-Aleksi Keller-Reichenbecher, Sandhausen (DE); Sebastian Schmidt, Weisendorf (DE)

(72) Inventors: David Grodzki, Erlangen (DE); Annemarie Hausotte, Erlangen (DE); Björn Heismann, Erlangen (DE); Arne Hengerer, Möhrendorf (DE); Mark-Aleksi Keller-Reichenbecher, Sandhausen (DE); Sebastian Schmidt, Weisendorf (DE)

(21) Appl. No.: 14/837,060

(22) Filed: Aug. 27, 2015

(30) Foreign Application Priority Data
Sep. 15, 2014 (DE) 102014218454.1

(51) Int. Cl. A61B 5/06; A61B 5/055; G06T 11/60; G06T 11/00; G06T 7/00; A61N 5/10; A61B 5/00

(52) U.S. Cl. CPC A61B 5/062; A61N 5/1001; A61B 5/055; A61B 5/7282; G06T 11/003; G06T 7/0042; G06T 11/60; G06T 2207/10088

(57) ABSTRACT

An apparatus is provided for obtaining a spatial instrument image of a medical instrument with a magnetic resonance tomography device, wherein the apparatus includes a medical instrument, a magnetic resonance tomography device, and a computing and control device. The medical instrument includes at least one marker material in at least one region, the marker material having a nuclear spin resonance outside of the proton resonance and wherein the computing and control device is configured to control the magnetic resonance tomography device such that a nuclear spin tomography imaging may be implemented by the magnetic resonance tomography device with the nuclear spin resonance of the at least one marker material in order to obtain a spatial instrument image, and wherein the computing and control device is configured to accept the instrument image. A corresponding medical instrument and a corresponding method are also provided.

### CROSS-REFERENCE TO RELATED APPLICATIONS

This application claims the benefit of DE 10 2014 218 454.1, filed on Sep. 15, 2014, which is hereby incorporated by reference in its entirety.

### TECHNICAL FIELD

The present embodiments relate to an apparatus for obtaining a spatial image of a medical instrument with a magnetic resonance tomography device. Furthermore, the present embodiments relate to a corresponding medical instrument and to a corresponding method for obtaining a spatial image of a medical instrument with a magnetic resonance tomography device.

### BACKGROUND

Magnetic resonance tomography, nuclear spin tomography, or in short MRT, is a known imaging method, which is used in medical diagnostics for representing structures and functions of soft tissue and organs in an examination object, e.g., a human or animal patient. Aside from many advantages over other imaging methods, such as computed tomography, x-ray imaging, or ultrasound imaging, magnetic resonance tomography imaging has the disadvantage that image objects having a low water or fat content are principally only very poorly imaged in a magnetic resonance tomography image, and are therefore difficult to identify. The position and location of a medical instrument or device in respect of other components, such as soft tissue, are in particular only apparent with difficulty in a magnetic resonance tomography image.

A field of application in which the information relating to the position of a medical instrument in relation to other objects such as organs is of huge importance is what is known as brachytherapy. Brachytherapy is a minimally invasive method of irradiating a tumor, (e.g., a prostate carcinoma, a cervical carcinoma, a mammary carcinoma, or a larynx carcinoma), by internal radiation therapy or radiation treatment in its immediate target region. To this end one or a number of radiation sources are positioned in close proximity to the region to be irradiated. One significant advantage over external beam radiotherapy, EBRT, is here if radioisotopes with a correspondingly short range are selected, such as is the case for instance with beta emitters, the radiation exposure for the surrounding tissue is minimal, whereas with external beam radiotherapy, healthy tissue also has to be penetrated in order to reach the target.

In order to introduce the radiation sources, so-called applicators or guides, (in other words catheter-type apparatuses or hollow needles), are frequently inserted or implanted into the body, close to the tumor or directly into the tumor tissue. With the so-called temporary brachytherapy, the radiation sources may survive in the body temporarily, (e.g., for a few minutes or hours), or in the case of permanent brachytherapy may survive in the body for a longer or unlimited period of time. With permanent brachytherapy, reference may also be made to low dose rate brachytherapy, LDR, and with temporary brachytherapy, since a more powerful radiation source is used to irradiate the tumor, reference may be made to high dose rate brachytherapy, HDR.

In order to determine the precise target position of the radiation source, a computed tomography (CT) or magnetic resonance tomography (MRT) recording of the region to be irradiated may be produced for instance prior to the therapy. The precise dose distribution in the target region is calculated on an irradiation planning system with the aid of this data record. The number and the positions of the applicators to be introduced and the radiation sources are determined on the basis of a dose distribution on or in the tumor. On account of the dose planning, the radiation is only applied with a high dose where the tumor is located. A dose distribution may also take place after implantation of the applicators and if necessary once again during the insertion of the radiation sources for quality control purposes. As a result the surrounding and in part most radiation-sensitive tissue is not unnecessarily irradiated and damage is minimized. Moreover, contrary to an external irradiation, the skin is not damaged since irradiation is performed from the inside.

The actual brachytherapy is performed following a preliminary examination, the dose planning and the acquisition of necessary materials. To this end, the patient is sedated or anesthetized in a sterile environment (OP) and the applicators are implanted. This may take place using 2D fluoroscopy. After successful control of the position of the applicators, the internal irradiation takes place with the aid of radioactive radiation sources, so-called seeds, e.g., in the form of approximately one to five millimeter long capsules made for instance of cesium-137. With the so-called afterloading method, the seeds are inserted manually or automatically through the applicators into their target region, if necessary in stages. The radiation dose in the target region is calculated by way of the radiation intensity of the individual seeds to be expected and their dwell time in the applicator or in the target region. If the forecast dwell time is reached, the seeds and the applicators are if necessary removed again in stages, in the case of a temporary brachytherapy. The dwell time and the calculated applied dose may be documented.

It is apparent that precise knowledge of the position of the applicators or the seeds with respect to other structures is required for a dose calculation. Nevertheless, a precise representation of the tumor and the surrounding organs at risk (OAR) is also important to be able to calculate a dose distribution both for the tumor volume and also for the organs at risk. Different imaging methods may be used here. In certain cases, computed tomography is used in current clinical practice since spatially-resolved 3D data records in which the applicators may be identified may be supplied therewith. The disadvantage of using computed tomography is that the target organs may only be delimited inadequately, e.g., in small pelvis minors. Magnetic resonance tomography would be suitable here, nevertheless with the disadvantage that applicators may now only be identified with difficulty. These are laboriously identified and segmented by a user, (e.g., a physician), in order to be able to consider them in a planning system. This disadvantage is so significant that magnetic resonance tomography was previously barely used for this application. Other failings of the magnetic resonance tomography, which play an important role in the dosimetry for EBRT methods, such as distortion, determination of attenuation values of the tissue, skin limits outside of the imaging region of the device, conversely hardly play any role in the brachytherapy because the target volume is close to the isocenter of the MR device. Only the direct environment of the tumor has to be considered and deviations in the radiation absorption barely carry any authority on account of the minimal range. The magnetic resonance tomography would therefore be well suited to carrying out dose calculations for the brachytherapy if the problem in terms of visibility of the applicators were to be resolved.

### SUMMARY AND DESCRIPTION

The scope of the present invention is defined solely by the appended claims and is not affected to any degree by the statements within this summary. The present embodiments may obviate one or more of the drawbacks or limitations in the related art.

An apparatus is provided for obtaining a spatial image of a medical instrument with a magnetic resonance tomography device. Furthermore, a corresponding medical instrument and a corresponding method are provided for obtaining a spatial image of a medical instrument with a magnetic resonance tomography device.

In certain embodiments, an apparatus is provided for obtaining a spatial image of a medical instrument with a magnetic resonance tomography device, wherein the apparatus includes a medical instrument, a magnetic resonance tomography device and a computing and control device, wherein the medical instrument includes at least one marker material in at least one region, the marker material having a nuclear spin resonance outside of the proton resonance, and wherein the computing and control device is embodied to control the magnetic resonance tomography device such that a nuclear spin tomography imaging with the nuclear spin resonance of the at least one marker material may be implemented by the magnetic resonance tomography device in order to obtain a spatial instrument image, and wherein the computing and control device is embodied to accept the instrument image.

The apparatus, with the aid of which a spatial image of a medical instrument, (e.g., a catheter), or at least part of the medical instrument may be obtained with a magnetic resonance tomography device. The apparatus includes the medical instrument, by which the spatial image may be obtained, a magnetic resonance tomography device and a computing and control device. The medical instrument includes at least one marker material in at least one region, the marker material having a nuclear spin resonance outside of the proton resonance. The computing and control device is configured to control the magnetic resonance tomography device and the computing and control device and the magnetic resonance tomography device are embodied such that a nuclear spin tomography imaging may be implemented by the magnetic resonance tomography device with the nuclear spin resonance of the at least one marker material in order to obtain a spatial instrument image. The computing and control device is embodied to accept the instrument image. The medical instrument thus includes a material, here called marker material, which indicates a nuclear spin resonance at a frequency that does not correspond to the proton resonance. Subsequently, regions having the marker material in a magnetic resonance tomography image, which was obtained with a conventional magnetic resonance tomography device, are not or are barely visible. In order to render the marker material visible in a magnetic resonance tomography image, referred to as the instrument image, the computing and control device controls the magnetic resonance tomography image such that a nuclear spin tomography imaging is enabled with the nuclear spin resonance of the marker material. To this end, high frequency radio frequency pulses are emitted in the magnetic resonance tomography device for instance by a radio frequency antenna unit by suitable antenna facilities and then radiated magnetic resonance signals are received and further processed by suitable radio frequency antenna.

The at least one marker material may include a fluorine compound, a sodium compound, and/or a phosphorus compound.

Fluorine compounds, 19F, (e.g., perfluorocarbons), may be used as marker material, since these only occur to a very minimal degree in the body. Other isotopes such as sodium, 21Na, or phosphorus, 31P and their compounds are however also conceivable as markers.

In an advantageous development, the medical instrument, aside from the at least one marker material, includes a transparency material, which has a nuclear spin resonance outside of the proton resonance and a marker material outside of the nuclear spin resonance of the at least one marker material.

By using a transparency material, the nuclear spin resonance of which neither corresponds to the proton resonance nor to the nuclear spin resonance of the at least one marker material, the transparency material is virtually transparent in an image, which is obtained with conventional parameters of a magnetic resonance tomography device and in an image obtained by a nuclear spin tomography imaging with the nuclear spin resonance of the at least one marker material. In particular, the medical instrument contains none or little material, which causes artifacts in the magnetic resonance tomography, in other words above all no metals or other electrically conductive materials. The transparency material may be a plastic.

In a further advantageous embodiment, the medical instrument includes the at least one marker material as a coating, as a compound, as an encapsulated enclosure, or at least one marker material is dissolved in a material of the medical instrument.

The marker material may be dissolved in a material of the medical instrument, (e.g., in a material of the outer shell of the medical instrument), or it may be contained as a so-called compound. For example, if the material is a transparency material, like plastic, the marker material may be mixed with the transparency material by simple manufacturing processes. By a marker material arranged or applied as a coating on at least one part of the outer shape of the medical instrument, the outer profile in the region of the marker material of the medical instrument may be made visible in the instrument image. An embodiment, in which a coating with plastics containing fluorine, (e.g., PTFE), trade name “Teflon”, may be applied to a medical instrument, (e.g., an applicator). This is advantageous in that the coating similarly positively influences the properties of the medical instrument, for instance bacteria or other coatings adhere more poorly to the medical instrument. If the medical instrument is a seed, this may be encased with a plastic, in which the marker material or the marker substance is contained. It is also conceivable for the marker material to be disposed in liquid form in suitable compartments in the medical instrument.

The computing and control device is particularly advantageously embodied to control the magnetic resonance tomography device such that a nuclear spin tomography imaging may be implemented by the magnetic resonance tomography device with the proton resonance in order to obtain a spatial anatomy image, and wherein the computing and control device is embodied to accept the anatomy image and to superimpose the anatomy image and the instrument image in a positionally correct and location correct manner.

A nuclear spin tomography imaging with the proton resonance is the conventional imaging with a magnetic resonance tomography device. It is thus possible by this feature to obtain an instrument image, to display the medical instrument, and an anatomy image, to display anatomical structures surrounding the medical instrument. This may take place successively or, through corresponding sequences, also simultaneously or almost simultaneously. The images may then be superimposed, fused, or registered. The image registration of two images refers to a method in digital image processing, with the aid of which two images of at least one similar scene are brought into agreement with one another in the best possible manner. In particular, if both images are obtained at the same time or within a short time interval from one another, the location from the medical instrument to the anatomical structures is provided, because both recordings are obtained with the same position of an examination object. As a result, a registration of the two images may be implemented easily.

The computing and control device may be configured to segment the medical instrument in the instrument image.

Segmentation is a common method in medical image processing. Segmentation may refer to the release of the medical instrument from other objects or image components not associated with the medical instrument. A simple segmentation method is for instance a threshold value method. Segmentation algorithms that appear useful to the person skilled in the art, for instance a region-growing algorithm, may be used for the segmentation of the medical instrument. Segmentation is easily possible, since the normal anatomical structures are not imaged in the instrument image, since these do not contain the marker substance or only contain it in tiny quantities and thus emit no signal.

It is proposed that as a function of the geometry and/or a deformability of the medical instrument, the medical instrument includes a number of predefinable regions of at least one marker material.

The markers with the marker material on the medical instrument may be positioned such that a position and a course of the medical instrument may be clearly determined from the instrument image. A rigid, needle-type applicator thus requires at least two markers at different positions for instance, wherein one marker may be disposed at the distal end of the applicator. With a flexible medical instrument, a number of markers may be distributed across the shape of the medical instrument, in order thus to be able to determine the position and course of the medical instrument from the instrument image. Alternatively, the entire medical instrument may naturally also be marked, e.g., by it being manufactured entirely from plastic, which indicates the desired resonance behavior or that the marker material is arranged in the form of a strip along the medical instrument. One marker is sufficient in the case of seeds.

A further advantageous embodiment provides that the at least one region with the at least one marker material has a predefinable geometry.

Certain structures that are particularly relevant to a correct positioning, for instance the ring in applicators for cervical carcinoma, may thus be marked.

In an alternative embodiment, the medical instrument includes a number of regions with different marker materials.

One marker may be marked or labeled for instance with a material, such as fluorine, 19F, the other with another material, such as sodium, 21Na. As a result, a similar number of images as marker materials used is obtained, however the information content also increases as a result, since different ends of the medical instrument may be marked with different marker materials for instance.

It is conceivable for the computing and control device to be configured to correct geometric distortions of the instrument image and/or the anatomy image from a known location and/or geometry of the at least one region with the at least one marker material.

One advantage with a number of markers is that if the distance between the markers is known, geometric distortion artifacts may be easily identified and at least partially compensated since an absolute standard criterion is available.

It has proven advantageous if an item of information is encoded on account of a geometric arrangement and/or the type of marker material and/or the number and/or the density of marker material of the at least one region with at least one marker material.

The markers may also be arranged in the manner of a bar code for instance such that the arrangement, the material or the quantity is specific to a certain type of medical instrument, e.g., an applicator, so that the medical instrument may be easily identified and mistakes may be ruled out. This may take place for instance by different distances between the markers or different marker materials being selected. Moreover the quantity or density of the material may vary, so that a distinction is possible with the aid of the measured signal intensity.

It is moreover advantageous if the computing and control device is configured to determine the position and location of the medical instrument by the instrument image and make a planning device available.

In many instances the local relation of a medical instrument to anatomical structures is of huge importance, for instance with neuronal surgical interventions. The position and location of the medical instrument may be determined by the instrument image and made available to a planning device, so that the planning device uses this information and may for instance determine a distance between a biopsy needle and a blood vessel.

The computing and control device favorably includes an image model of the medical instrument and the computing and control device is configured to determine the position and the location of the medical instrument by the instrument image and to superimpose the image model of the medical instrument with the anatomy image in a positionally correct and location correct manner.

Alternatively to superimposing an anatomy image with image data of the instrument image, digital image processing methods may be used to determine the position and alignment of the medical instrument in the image space and an image model, (e.g., a simplified representation of the medical image), is superimposed onto the anatomy image. The image model of the medical instrument may be embodied in detail in a predefinable manner. The medical instrument may for instance be modeled in a realistic or abstract manner on account of the known ray tracing method, (e.g., solely by an arrow), which indicates the alignment and a distal end. In one embodiment, the position and alignment of an applicator may be indicated schematically to a customer, (e.g., as a point with an appended line), in real-time in the superimposition for an anatomical representation. The type of applicator may also be determined from a previously described identifier and represented by way of example.

The medical instrument is expediently an applicator for implementing a brachytherapy, or a radiation device for use in a brachytherapy.

As apparent from the preceding embodiments, the described apparatuses are particularly suited to obtaining a spatial image with a magnetic resonance tomography device of an applicator or a radiation device for use in brachytherapy.

In certain embodiments, a medical instrument is provided for obtaining a spatial image of a medical instrument with a magnetic resonance tomography device, wherein the medical instrument may be used to obtain a spatial image of a medical instrument with a magnetic resonance tomography device, if it is configured like one of the afore-described medical instruments and is described with one of the afore-described apparatuses.

Such a medical instrument for obtaining a spatial image of a medical instrument with a magnetic resonance tomography device in at least one region includes for instance at least one marker material, which has a nuclear spin resonance outside of the proton resonance. Together with an apparatus that includes a magnetic resonance tomography device and a computing and control device, wherein the computing and control device is embodied to control the magnetic resonance tomography device such that a nuclear spin tomography imaging may be implemented by the magnetic resonance tomography device with the nuclear spin resonance of the at least one marker material, a spatial instrument image of the medical instrument may be obtained, which may be accepted by the computing and control device.

A method is provided for obtaining a spatial image of a medical instrument with a magnetic resonance tomography device, wherein the method uses one of the previously described apparatuses to obtain a spatial image of a medical instrument with a magnetic resonance tomography device.

In such cases, the method includes acts for the purpose of which components of the apparatus may be configured. With a marker material, which has a nuclear spin resonance outside of the proton resonance and a computing and control device, which is embodied to control a magnetic resonance tomography device such that a nuclear spin tomography imaging may be implemented by the magnetic resonance tomography device with the nuclear spin resonance of the at least one marker material in order to obtain a spatial instrument image, a method act may read: Control the magnetic resonance tomography device by the computing and control device such that a nuclear spin tomography imaging is implemented by the magnetic resonance tomography device with the nuclear spin resonance of the at least one marker material and obtain a spatial instrument image.

An example of a method is described below in a brachytherapy. With the brachytherapy, after positioning the applicators, at least one recording with proton resonance, the anatomy image and at least one recording with the at least one resonance frequency of the marker substance, the instrument image, is made. This may take place successively or also at the same time. The applicators are then automatically segmented in the instrument image. This is less complicated, since the normal anatomical structures are not imaged in this recording, since these do not contain the marker substance or only contain it in tiny quantities and thus emit no signal. The location relative to the anatomical structures is however consequently guaranteed, because both recordings are made without moving the patient in the same position. The positions or courses of the applicators that are determined in such a way are then taken into account when planning the brachytherapy. To this end, they are depicted in the representation overlying the anatomical image and highlighted in color for instance.

### BRIEF DESCRIPTION OF THE DRAWINGS

Further advantageous developments result from the following figures and description.

FIG. 1 depicts a representation to describe a brachytherapy according to the prior art.

FIG. 2 depicts a schematic and exemplary representation of a medical instrument for obtaining a spatial image of a medical instrument with a magnetic resonance tomography device.

FIG. 3 depicts a schematic and exemplary representation of an instrument image, an anatomy image, and a superimposition of the two images.

FIG. 4 depicts a schematic and exemplary representation of an apparatus for obtaining a spatial image of a medical instrument with a magnetic resonance tomography device.

### DETAILED DESCRIPTION

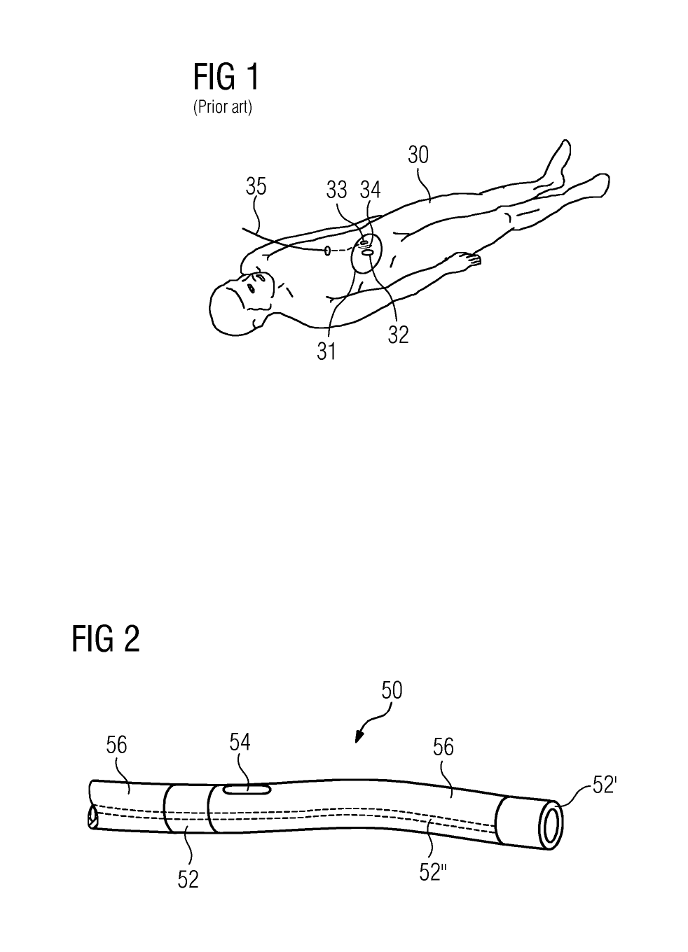

FIG. 1 depicts a representation to describe a brachytherapy according to the prior art. With an examination object 30, here a human patient, a brachytherapy is implemented to treat a target object 32, here a tumor. To this end, an applicator 35, here in the form of a catheter, is inserted into a target region 31. The target region 31 includes at least the target object 32, (e.g., the target region may be a volume within the examination object 30), within which at least the target object 32 is disposed. The applicator 35 allows a radiation device 33, here a so-called seed made of radionuclide, for instance cesium-137, cobalt-60, iridium-192, iodine-125, palladium-103 or ruthenium-106, or also a miniaturized low-energy x-ray emitter, to be brought into the immediate vicinity of the target object 32.

The radiation device 33 emits high-energy beams, indicated by lines 34 in FIG. 1, which penetrate the target object 32. A significant advantage of brachytherapy is that the radiation effect relates to a very limited area around the radiation source. Consequently, tissue and organs in the environment of the radiation device 33 are also irradiated so that the location of the introduction and the type of radiation device 33 and the duration of the treatment have to be very well considered in order to minimize the health risks to the examination object 30.

In FIG. 2, a medical instrument 50, here a catheter, for obtaining a spatial image of a medical instrument 50 with a magnetic resonance tomography device, is depicted schematically and by way of example. The medical instrument 50 thus includes a material in a number of regions 52, 52′, 52″, 54, here called marker material, which indicates a nuclear spin resonance at a frequency that does not correspond to the proton resonance. Subsequently, regions 52, 52′, 52″, 54, which have the marker material in a magnetic resonance tomography image, which was obtained with a conventional magnetic resonance tomography device, are not or are barely visible.

In order to render the marker material visible in a magnetic resonance tomography image, known as the instrument image, a computing and control device controls the magnetic resonance tomography image such that a nuclear spin tomography imaging is enabled with the nuclear spin resonance of the marker material.

The medical instrument 50 indicated in FIG. 2 has a cuff-type region 52, an annular region 52′, a strip-type region 52″ and a region 54 with marker material. The region 54 is characterized in that an item of information is encoded by the special geometric form, for instance the type of medical instrument 50.

The medical instrument 50 further includes regions 56, which include a so-called transparency material. By using a transparency material, the nuclear spin resonance of which neither corresponds to the proton resonance nor to the nuclear spin resonance of the at least one marker material, the transparency material is virtually transparent in an image obtained with conventional parameters of a magnetic resonance tomography device and in an image obtained by a nuclear spin tomography imaging with the nuclear spin resonance of the at least one marker material.

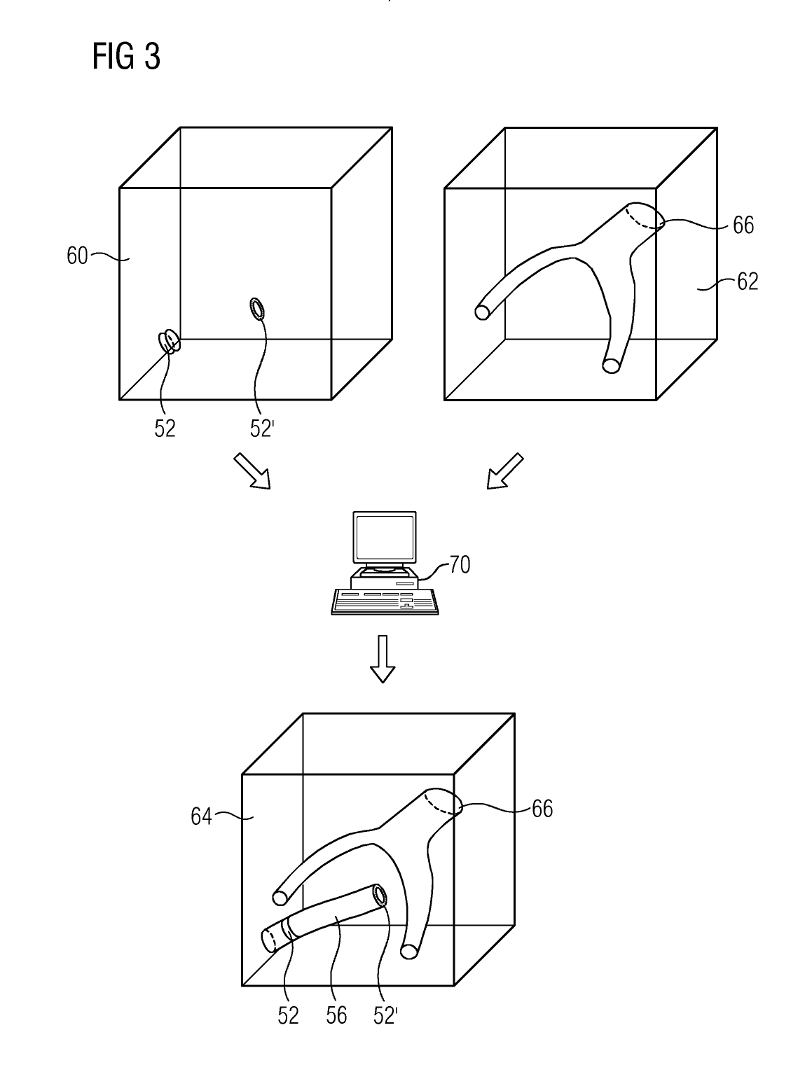

FIG. 3 depicts a schematic and exemplary representation of an instrument image 60, an anatomy image 62, and a superimposition of the two images 64. One of the afore-described apparatuses is configured to obtain a spatial instrument image 60 of a medical instrument with a magnetic resonance tomography device. To this end, the apparatus includes the medical instrument, the magnetic resonance tomography device and a computing and control device 70, wherein the medical instrument includes a marker material in regions 52 and 52′, which includes a nuclear spin resonance outside of the proton resonance and wherein the computing and control device 70 is embodied to control the magnetic resonance tomography device such that a nuclear spin tomography imaging may be implemented by the magnetic resonance tomography device with the nuclear spin resonance of the at least one marker material in order to obtain the spatial instrument image 60. The regions 52 and 52′ or the markers with the marker material on the medical instrument are positioned such that a position and a course of the medical instrument may be clearly determined from the instrument image 60. In this exemplary embodiment, a rigid catheter, the two regions are arranged at different positions, the region 52′ at the distal end of the applicator and the other region 52 on the shaft of the catheter.

The computing and control device 70 is in this exemplary embodiment also embodied to control the magnetic resonance tomography device such that a nuclear spin tomography imaging may be implemented by the magnetic resonance tomography device with the proton resonance in order to obtain the spatial anatomy image 62 of an anatomical structure 66, here a vessel. The computing and control device 70 accepts the instrument image 60 and the anatomy image 62 from the magnetic resonance tomography device. The computing and control device 70 determines the location and the position of the medical instrument by methods of digital image processing that are known per se from the instrument image 60 and with this information superimposes an image model 56 of the medical instrument onto the anatomy image 62 in a positionally and location correct manner, as a result of which a superimposition image 64 is produced. The superimposition image 64 may for instance be displayed on a representation, e.g., a computer monitor. The position and location of the medical instrument may also be made available to a medical planning device or a documentation device, so that this may use this information further.

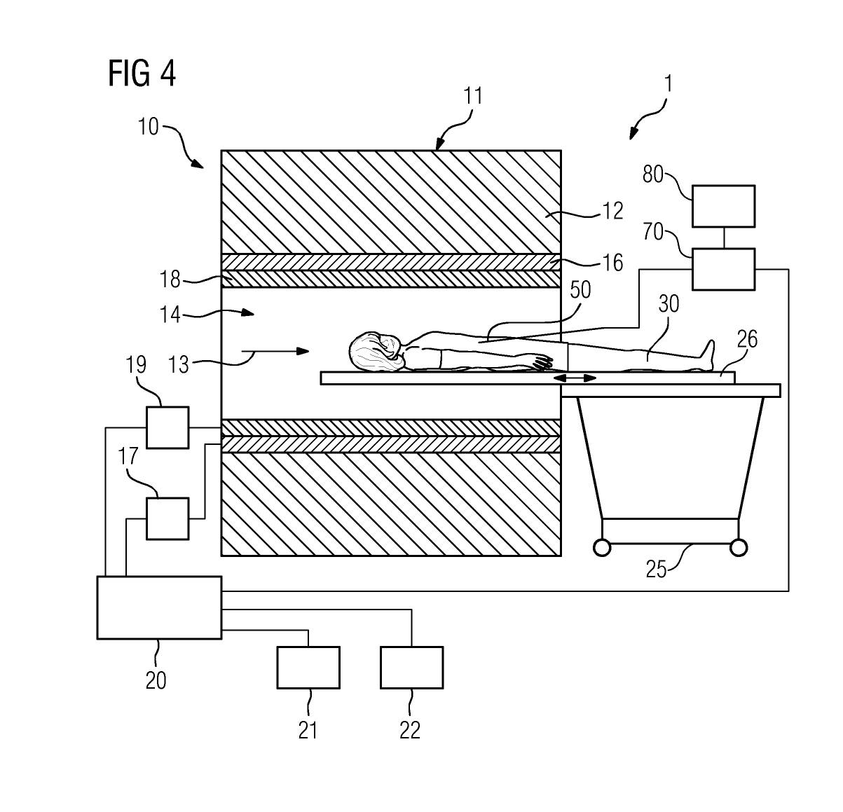

In FIG. 4, an apparatus 1 for obtaining a spatial image of a medical instrument 50 with a magnetic resonance tomography device 10 is depicted schematically and by way of example. The magnetic resonance tomography device 10 includes a magnet unit 11 having a superconducting main magnet 12 for generating a powerful and in particular constant main magnetic field 13.

Moreover, the magnetic resonance apparatus includes a patient receiving zone 14 for receiving an examination object 30, here a human patient. The patient receiving zone 14 in the present exemplary embodiment is embodied in a cylindrical design and is surrounded cylindrically in a peripheral direction by the magnet unit 11. An embodiment of the patient receiving zone 14 that deviates therefrom is however conceivable at any time. The examination object 30 may be pushed into the patient receiving zone 14 by a patient support apparatus 25 of the magnetic resonance tomography device 10. The patient support apparatus 25 to this end has a couch 26 configured to be movable within the patient receiving zone 14.

The magnet unit 11 also has a gradient coil unit 16 for generating magnetic field gradients that are used for local encoding during imaging. The gradient coil unit 16 is controlled by a gradient control unit 17 of the magnetic resonance tomography device 10. The magnet unit 11 furthermore has a radio frequency antenna unit 18 for exciting a polarization that develops in the main magnetic field 13 generated by the main magnet 12. The radio frequency antenna unit 18 is controlled by a radio frequency antenna control unit 19 of the magnetic resonance tomography device 10 and radiates radio frequency magnetic resonance sequences into an examination space that is substantially formed by a patient receiving zone 14 of the magnetic resonance tomography device 10.

In order to control the main magnet 12, the gradient coil unit 17 and in order to control the radio frequency antenna control unit 19, the magnetic resonance apparatus has a control unit 20. The control unit 20 centrally controls the magnetic resonance apparatus, such as performing a predetermined imaging gradient echo sequence for example. Moreover, the control unit 20 has an evaluation unit for evaluating image data.

Control information such as imaging parameters, for example, as well as reconstructed magnetic resonance images may be displayed on a display unit 21, for example on at least one monitor, of the magnetic resonance tomography device 10 for viewing by an operator. Furthermore the magnetic resonance tomography device 10 has an input unit 22 by which information and/or parameters may be input by an operator during a measurement procedure.

The magnetic resonance tomography device 10 disclosed may naturally include further components not further disclosed herein. A general method of functioning of a magnetic resonance tomography device is also known to a person skilled in the art, so that a detailed description of the further components is not included.

The apparatus 1 for obtaining a spatial image of a medical instrument 50 with a magnetic resonance tomography device 10 includes the medical instrument 50, here an applicator for implementing a brachytherapy, the magnetic resonance tomography device 10 and a computing and control device 70, for instance a computer. The medical instrument 50 includes at least one marker material in at least one region, the marker material having a nuclear spin resonance outside of the proton resonance.

The computing and control device 70 is configured to control the magnetic resonance tomography device 10 and the computing and control device 70 and the magnetic resonance tomography device 10 are embodied such that a nuclear spin tomography imaging may be implemented by the magnetic resonance tomography device 10 with the nuclear spin resonance of the at least one marker material in order to obtain a spatial instrument image. The computing and control device 70 is embodied to accept the instrument image, and to this end includes a connecting device, here an electrical line, to the control unit 20 of the magnetic resonance tomography device 10.

The medical instrument 50 thus includes a marker material, which indicates a nuclear spin resonance at a frequency that does not correspond to the proton resonance. Subsequently, regions that have the marker material in a magnetic resonance image, which was obtained with a conventional magnetic resonance tomography device 10, are not or are barely visible. In order to render the marker material visible in a magnetic resonance tomography image, the computing and control device 70 controls the magnetic resonance tomography device 10 such that a nuclear spin tomography imaging is enabled with the nuclear spin resonance of the marker material. To this end, high frequency radio frequency pulses are emitted in the magnetic resonance tomography device 10 by the high frequency antenna unit 18 by suitable antenna facilities and then radiated magnetic resonance signals are received and further processed by suitable radio frequency antenna.

The computing and control device 70 may also be integrated in the magnetic resonance tomography device 10, e.g., in the control unit 20. Furthermore, the computing and control device 70 may be configured to determine the position and location of the medical instrument 50 by the instrument image and to forward the same to a planning device 80, here a planning system for brachytherapy. The medical instrument 50 may be embodied by a geometric arrangement, by the type of marker material, by the number or the density of marker material with the at least one region of the medical instrument 50, carry a piece of information that may be decoded by the computing and control device 70 and be displayed on the display unit 21 for instance.

In summary, further embodiments and advantages are described. The embodiments propose inter alia an apparatus, which allows for an automatic identification of brachytherapy applicators in nuclear spin resonance tomography. To this end, markers with a corresponding substance and nuclear spin resonance tomography with another frequency are used as the proton imaging.

The simple and fault-free identification of the location of the applicators relative to the anatomy is advantageous. Even with poor image quality, the applicators may be well identified if an isotope, (e.g., fluorine), is used, which only occurs to a very minimal degree in the body.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An apparatus for obtaining a spatial instrument image of a medical instrument with a magnetic resonance tomography device, the apparatus comprising:
a medical instrument;
a magnetic resonance tomography device; and
a computing and control device,
wherein the medical instrument comprises at least one marker material in at least one region, the at least one marker material having a nuclear spin resonance outside of a proton resonance,
wherein the computing and control device is configured to control the magnetic resonance tomography device such that a nuclear spin tomography imaging is configured to be implemented by the magnetic resonance tomography device with the nuclear spin resonance of the at least one marker material in order to obtain a spatial instrument image, and
wherein the computing and control device is configured to accept the instrument image.

2. The apparatus as claimed in claim 1, wherein the at least one marker material comprises fluorine, sodium, phosphorous, or a combination thereof.

3. The apparatus as claimed in claim 1, wherein the medical instrument further comprises a transparency material having a nuclear spin resonance outside of the proton resonance and outside of the nuclear spin resonance of the at least one marker material.

4. The apparatus as claimed in claim 3, wherein the medical instrument further comprises a number of predefinable regions of the at least one marker material as a function of a geometry, a deformability, or the geometry and the deformability of the medical instrument.

5. The apparatus as claimed in claim 4, wherein the at least one marker material comprises different marker materials.

6. The apparatus as claimed in claim 1, wherein the at least one marker material is a coating, a compound, or an encapsulated encasing of the medical instrument, or
wherein the at least one marker material is dissolved in a material of the medical instrument.

7. The apparatus as claimed in claim 1, wherein the nuclear spin tomography imaging is configured to be implemented by the magnetic resonance tomography device with the proton resonance in order to obtain a spatial anatomy image, and
wherein the computing and control device is configured to accept the anatomy image and to superimpose the anatomy image and the instrument image in a positionally correct and location correct manner.

8. The apparatus as claimed in claim 7, wherein the computing and control device is configured to correct geometric distortions of the instrument image, the anatomy image, or the instrument and anatomy images from a known location, geometry, or location and geometry of the at least one region with the at least one marker material.

9. The apparatus as claimed in claim 1, wherein the computing and control device is configured to segment the medical instrument in the instrument image.

10. The apparatus as claimed in claim 1, wherein the medical instrument further comprises a number of predefinable regions of at least one marker material as a function of a geometry, a deformability, or the geometry and the deformability of the medical instrument.

11. The apparatus as claimed in claim 10, wherein the at least one marker material comprises different marker materials.

12. The apparatus as claimed in claim 1, wherein the at least one region with the at least one marker material has a predefinable geometry.

13. The apparatus as claimed in claim 1, wherein the medical instrument comprises a number of regions with different marker materials.

14. The apparatus as claimed in claim 1, wherein the computing and control device is configured to correct geometric distortions of the instrument image, an anatomy image, or the instrument and anatomy images from a known location, geometry, or location and geometry of the at least one region with the at least one marker material.

15. The apparatus as claimed in claim 1, wherein an item of information is encoded with the at least one marker material on account of one or more of the following: a geometric arrangement, a type of marker material, a number of marker materials, or a density of the at least one marker material of the at least one region.

16. The apparatus as claimed in claim 1, wherein the computing and control device is configured to: (1) determine a position and a location of the medical instrument by the instrument image and (2) make a planning device available.

17. The apparatus as claimed in claim 1, wherein the computing and control device comprises an image model of the medical instrument, and wherein the computing and control device is configured to: (1) determine a position and a location of the medical instrument by the instrument image and (2) superimpose the image model of the medical instrument onto an anatomy image in a positionally and location correct manner.

18. The apparatus as claimed in claim 1, wherein the medical instrument is an applicator for implementing a brachytherapy or a radiation device for use in a brachytherapy.

19. A medical instrument comprising:
at least one marker material in at least one region, the at least one marker material having a nuclear spin resonance outside of a proton resonance,
wherein the medical instrument is configured to obtain a spatial instrument image of the medical instrument with a magnetic resonance tomography device.

20. The method for obtaining a spatial instrument image of a medical instrument with a magnetic resonance tomography device, the method comprising:
obtaining a spatial instrument image of a medical instrument with a magnetic resonance tomography device of an apparatus.
wherein the medical instrument comprises at least one marker material in at least one region, the at least one marker material having a nuclear spin resonance outside of a proton resonance,
wherein a computing and control device of the apparatus is configured to control the magnetic resonance tomography device such that a nuclear spin tomography imaging is configured to be implemented by the magnetic resonance tomography device with the nuclear spin resonance of the at least one marker material in order to obtain the spatial instrument image, and
wherein the computing and control device is configured to accept the instrument image.
